(19) United States
(12) Patent Application Publication
(10) Pub. No.: US 2008/0249391 A1
(43) Pub. Date: Oct. 9, 2008

(54) Chronic in-vivo neurotransmitter sensor

(75) Inventors: Karen A. Moxon, Collingswood, NJ (US); Dennis Litwin, Wilmington, DE (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Drexel University

(21) Appl. No.: 11/879,616

(22) Filed: Jul. 18, 2007

Related U.S. Application Data
(60) Provisional application No. 60/807,700, filed on Jul. 18, 2006.

(51) Int. Cl. A61N 1/04

(52) U.S. Cl. 600/373

(57) ABSTRACT

Carbon-coated ceramic based electrode arrays having a ceramic substrate patterned with multiple recording sites are provided. Potentiostat devices having said carbon-coated ceramic based electrodes, and methods of use, are also provided. Certain embodiments of the present inventive articles, devices, and methods are especially suited for detection and/or measurement of electroactive species.

### CROSS-REFERENCE TO RELATED APPLICATIONS

This application is entitled to priority under 35 U.S.C. §119(e) to U.S. Provisional Patent Application No. 60/807,700, filed Jul. 18, 2006, which is incorporated by reference herein in its entirety.

### BACKGROUND OF THE INVENTION

Neurotransmitters play an integral part in the transmission and modulation of neural signals in the brain. Neurotransmitter levels and fluctuations correlate with normal and pathological mood, behavior, and basic functioning of the central and peripheral nervous system. High speed, sensitive measurement of these levels in the living brain would be extremely valuable to drug and behavioral research.

For example, norepinephrine (NE), a neurotransmitter with cell bodies lying in the locus coeruleus (LC) and whose axons have diffuse projections throughout the brain, is implicated in mammalian sensory gating and attentional state. The locus coeruleus is the source of a diffuse network of norepinephrine-containing axons that project to multiple brain regions including the forebrain, cerebellum, brainstem and spinal cord. In certain animal experiments, it has been shown that as locus coeruleus output (i.e., norepinephrine) increases, performance on a discrimination task improves to an optimal state; but if locus coeruleus output continues to increase, performance will eventually degrade. Drugs that either block the uptake of norepinephrine, or modulate its release, will likely effect the performance on behavioral discrimination tasks. Indeed, certain drugs that ameliorate attentional deficit disorders symptoms have been shown to effectively block uptake sites for norepinephrine.

Because of its broad efferent projection, the LC-NE system has been still further implicated in a variety of global functions including sleep/arousal, learning/memory, sensory perception and cognition. Disruption of local norepinephrine concentration, however, has also been implicated in several mental illnesses including depression, schizophrenia and hyperactive disorders. Interestingly, recent studies suggest that blocking norepinephrine reuptake does not simply increase the extracellular concentration of norepinephrine throughout the brain but can have multiple, and varied, effects and may result in heterogeneous concentration of norepinephrine within various distant regions of the brain.

Although norepinephrine is implicated in a variety of brain functions, the precise mechanisms through which cells and circuits are influenced to alter behavioral responses remains unclear. Improved detection and measurement of norepinephrine would lead to greater understanding of certain attentional and behavioral brain states. For example, detection and measurement of the different distributions of extracellular norepinephrine concentration throughout the brain would be invaluable; particularly the spatial and temporal distribution of norepinephrine during locus coeruleus output over time. However, such measurements are currently highly problematic.

Neurotransmitter sensor technology already known in the art does not provide the capabilities for real time, in-vivo detection of extracellular norepinephrine with the required spatial mapping resolution. Indeed, there is a need in the art for electrochemical sensors, such as neurotransmitter sensors, that may simultaneously record the local and global (e.g., micro- and macro-) electrophysiological and electrochemical response of single neurons in certain brain regions of interest. Still further, increased ability to accurately determine neurotransmitter levels at lower concentrations and over shorter time intervals may also advance the understanding of the effects of certain neurotransmitter-blocking drug candidates. There is presently a need in the art for improved detection and measurement of neurotransmitters, for example and without limitation: dopamine, norepinephrine and serotonin, during active brain signaling.

Neurotransmitter levels are most traditionally determined by methods such as microdialysis, amperometry, or cyclic voltammetry. Microdialysis generally exhibits acceptable specificity regarding the signal generating electroactive chemical species but requires analysis times on the order of several seconds, or even several minutes, and requires separate complex analytical instrumentation that resists miniaturization and in-vivo implantation. Microdialysis, and associated high pressure liquid chromatography coupled to electrochemical detection (HPLC-EC), is a common method of studying changes in neurotransmitter concentration in target locations and drug modulated neurotransmitter release or reuptake. Microdialysis is, however, simply unable to resolve several physiological processes of interest since these processes operate on much shorter time intervals than microdialysis' required analysis time. Microdialysis also lacks spatial and temporal responsiveness required to properly simultaneously investigate micro and macro neurotransmitter levels during various brain activity levels over time. Traditional cyclic voltammetry and amperometry are also deficient as each lacks specificity regarding electroactive compound identification.

Still further, while many different types of electrodes have been designed for recording and stimulating mammalian central nervous system tissue, traditional chronic recording electrodes consist of a gold, platinum or stainless steel wire coated with an insulating material, except at the tip. These electrodes suffer drawbacks that reduce their usefulness as neural interface devices. These drawbacks include a low recording site (RS) to neuronal tissue displaced (NTD) ratio, difficulty of integrating on-board electronics sufficiently close to the electrode to reduce noise, and the inability to produce quality microwire electrodes for neural recording using batch processing.

These previously known electrodes are generally made by hand, resulting in considerable variation in the recording characteristics of each electrode tip. Further, a wide range of electrical characteristics of the electrode results in difficulties properly matching impedances with on-board electronics. The Ceramic Based Multi-Site Electrode arrays disclosed in U.S. Pat. No. 6,834,200 to Moxon et al., incorporated herein by reference, provide a significant advantage over conventional electrode technology.

Further improved multi-site electrochemical recording electrodes, and methods of use, would facilitate enhanced temporal and spatial resolution required to understand how neurotransmitter production, and reuptake, effect neural activity. Indeed, rapid detection and measurement of the local and global concentration of neurotransmitters and the neural activity of single neurons may provide a vastly improved picture of the effects of therapeutic intervention of signal processing in the brain.

### SUMMARY

A ceramic based multi-site electrode array having a polished ceramic substrate patterned with at least one recording site and at least one bonding pad which are connected via at least one conducting line, and an inert ceramic insulating layer encasing said conducting line is provided. The recording site is not encased by said insulating layer, but rather, is coated with at least one layer of carbon. Methods for detecting and/or measuring neurotransmitter concentration in at least two locations of a mammal brain are facilitated by certain embodiments of the present invention. Multi-channel potentiostat systems having at least one carbon coated ceramic based multi-site electrode array, and methods of using same, are also provided.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings certain embodiments which are presently preferred. It should be understood, however, that the invention is not limited to the precise arrangements and instrumentalities shown.

FIG. 1 represents schematic drawings of carbon-coated ceramic based multi-site electrode (CCBMSE) array embodiments of the present invention.

FIG. 2 depicts a graph showing norepinephrine concentration as a function of time.

FIG. 3 depicts neural signals simultaneously recorded from four recording sites on the CCBMSE array. Recordings were made from CCBMSE arrays chronically implanted into the somatosensory cortex of rats.

FIG. 4 depicts graphs showing dopamine concentration as a function of time and location.

FIG. 5 depicts a graph showing norepinephrine concentration pre- and post-administration of a norepinephrine-uptake blocking drug.

FIG. 6 depicts a graph showing relative sensitivities measured from exemplary CCBMSE arrays having varied recording site sizes (small and large) compared to a platinum electrode and three different sizes of commercially available carbon fiber electrodes.

FIG. 7 depicts a graph showing sensitivities measured from exemplary CCBMSE arrays having sputter deposited or pyrolysis deposited carbon recording site, as compared to conventional electrodes known in the art.

FIG. 8 depicts a schematic diagram of an exemplary Flow Injection Chamber used to measure the responsiveness of exemplary electrodes and for comparisons of response times to carbon fiber electrodes.

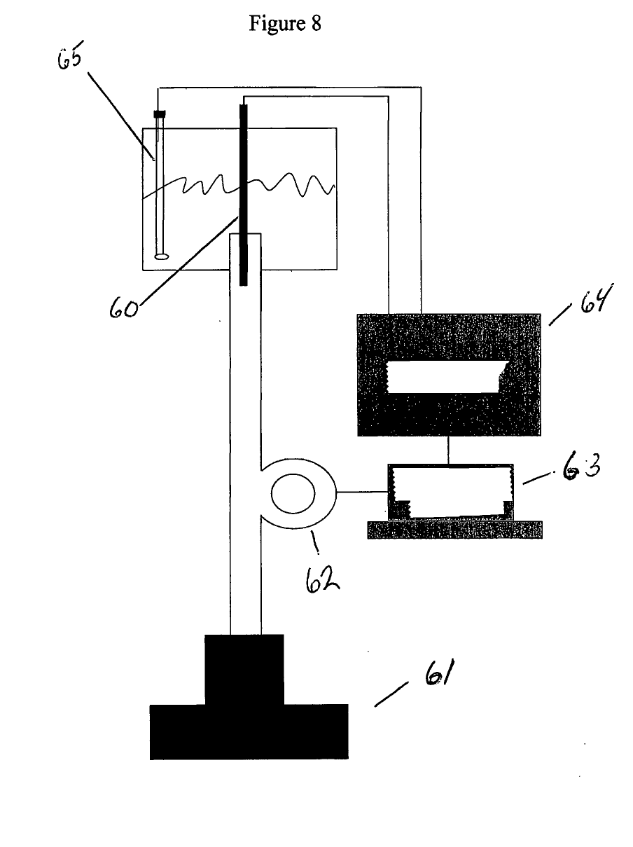

### DETAILED DESCRIPTION OF THE INVENTION

The present invention generally relates to methods of detection and measurement of electroactive compounds. Certain embodiments of the present invention relate to multi-electrode arrays and methods of producing and using same.

### Multi-Site Electrode Arrays

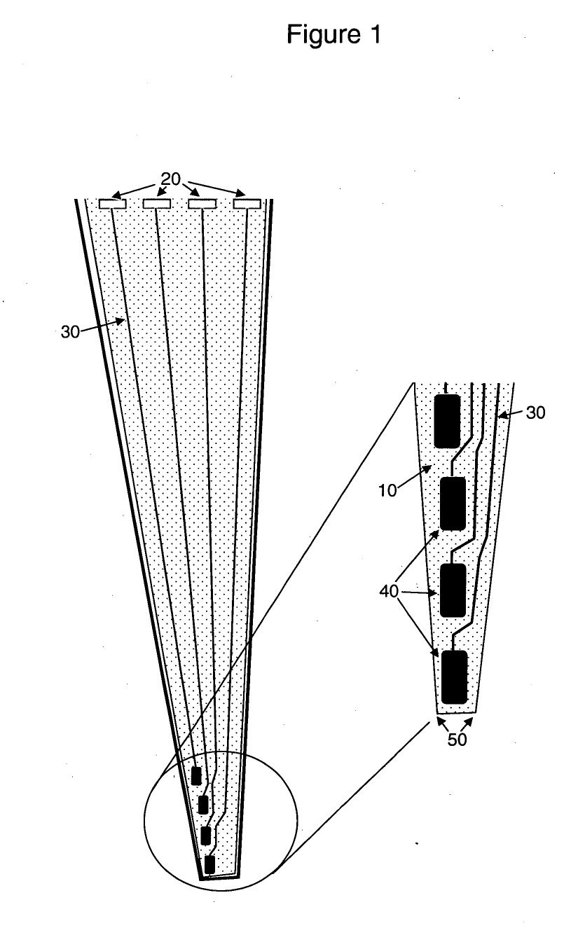

FIG. 1 depicts a carbon-coated ceramic based multi-site electrode (CCBMSE) embodiment of the present invention. The substrate 10 of the electrode is made of ceramic material that is generally non-reactive in the brain environment. It is relatively strong and rigid, which aids in inserting the electrode through the dura into deep brain structures. In an exemplary embodiment, the recording sites 40, conducting lines 30 and bonding pads 20 are patterned directly on to a ceramic substrate. In certain embodiments such patterning is facilitated by reverse photolithography thereby allowing for improved precision regarding the resist wall angles and resolution of features applied to the substrate. The CCBMSE arrays of the present invention generally comprise a polished ceramic substrate, preferably less than or equal to about 50 μm in thickness with a range of about 35 to about 50 μm being preferred, having a narrow tip at one end ranging up to about 0.1 mm in width and preferably pointed to cut through neural tissues, and a wider region at the opposite end approximately twice the width of the narrow tip. The wider region contains the bonding pads, does not enter the neural tissue and is sized to be large enough to provide contacts for on-board electronics such as VLSI. The polished ceramic substrate is patterned with multiple recording sites, preferably 2 to 32 recording sites, at the narrow tip of the substrate. Each recording site is connected to a bonding pad placed at the opposite wider end of the substrate via a conducting line which runs from the recording site to the bonding pad.

In exemplary embodiments, shown in FIG. 1, the CCBMSE arrays have four recording sites 40, spaced about 200 um apart, beginning about 60 um from the tip 50. The conducting lines are 8 um wide and features, such as recording sites 40, conducting lines 30 and bonding pads 20, are separated by at least about 5 μm. The electrode design is patterned onto Al₂O₃ (alumina) substrates purchased from Valley Design (Westford, Mass.). Preferably, such electrodes are patterned on to at least one 99.6% Al₂O₃ (alumina) substrate. Certain embodiments of the present invention may utilize any number of ceramic substrate materials, such as silicon; such alternate ceramic substrate material preferably has at least a portion of its surface coated by alumina.

In producing conducting surfaces such as bonding pads 20, conducting lines 30 and recording sites 40, a substrate wafer 10 is cleaned using a trichloroethylene 10-minute soak, followed by an acetone then methanol rinse, and dried by methanol vapor. To create the resist structures, substrate wafer 10 is mounted and spin-coated with photoresist (S1827, Shipley Co., Marlborough, Mass.) for about 20 seconds at about 3000 RPM with a about 21″ Hg pull down vacuum. The wafer is then hot plate baked at about 110° C. for about 60 seconds. Using an image reversal mask to produce a negative sidewall angle in the resist structure, the wafer is exposed with UV light (90 mj) and then baked with ammonia gas for about 1 hour at about 90° C. The wafer is then flood exposed with UV light at about 2,760 mj and developed for about 25 seconds.

Once the wafer is prepared with appropriate resist features defining the electrode pattern, it is cleaned, and a metal layer consisting of about 200 angstroms (Å) layer of chromium, followed by an about 1500 Å layer of platinum, is deposited. Excess deposited metal, along with the underlying photoresist, may be removed using acetone and MeOH followed by a N₂ blow dry. The metal layer defines recording sites 40, conducting lines 30 and the bonding pads 10 of the electrode.

The CCBMSE arrays are insulated with Al₂O₃ to encase the conducting lines in a layer of ceramic. The resulting sidewall angle of the metal layer features allow this subsequent insulation layer to adhere closely to the contours of the metal features. A second photomask leaves only the recording sites and the bonding terminals exposed. Photoresist is applied (spin-coat for about 20 seconds) over the entire circuit and developed using this photomask so that the terminals and bonding pads are protected, similar to the image reversal procedure described above. The substrates are then coated with an about 1000 Å layer of Al₂O₃, preferably using ion beam-assisted deposition. The resist over the recording sites and bonding terminals is removed, removing the unwanted insulation with acetone and MeOH with a N₂ blow dry. This leaves the recording sites and bonding pads exposed and the conducting lines insulated.

Bonding terminals are then attached to a micro-connector (Omnetics Inc., Minneapolis, Minn.) using thermo-sonic wire bonding. The connector is preheated to about 135° C., and an about 25.4 μm thick gold wire is used. The wire is extended from the bonding pad on the electrode to the gold tab on the connector. Total distance is about 0.2-about 1.0 mm. The output leads are separated by about 350 μm. The leads are then covered with a thick layer of non-conducting epoxy to protect them during an implantation procedure. The bonding sites preferably remain above the level of the skull during any such implantation procedure.

Following the insulation procedure, a carbon layer is applied to the recording site, i.e., recording tip, of the electrode. Such carbon deposition may be carried out by sputter deposition, ion-beam assisted deposition, or pyrolysis, or combinations thereof. Without being limited by theory, such carbon-coated ceramic based electrodes are believed to better detect and measure rapid transient changes in local neurotransmitter concentration.

In certain embodiments of the present invention, an about 2500 Å thick carbon film is sputter deposited onto the patterned substrate using a Perkin Elmer Model 2400 dc magnetron sputtering system. For certain embodiments, an about 50 Å layer of titanium is deposited on the insulation layer prior to carbon deposition thereby facilitating improved adhesion. The sputtering system is pumped down to a base pressure of about 3 μTorr, and argon gas is introduced to maintain a pressure of about 20 μTorr during deposition. The carbon is applied at a deposition rate of about 21 Å/min. The unwanted carbon overlying the photoresist is removed in a final lift-off step in boiling Nophenol 922 at about 90±5 degrees C.

Relatively rough carbon surfaces may improve the sensitivity of the electrodes by increasing the effective surface area for oxidation/reduction and increasing adsorption of electroactive species, e.g., monoamine neurotransmitters, during in-vivo operation. Generally speaking, a rough surfaced electrode may have better sensitivity than a smooth surface given the same electrode dimensions. However, the increased adsorption of electroactive species may reduce the ability of the electrode to respond to rapid transient concentration changes. In contrast, a relatively smooth carbon surface may facilitate larger electrode dimensions than a rough surfaced electrode since current transfer is proportional to electrode surface area. A larger electrode, however, leads to greater potential for damage to in-vivo neural tissue.

Given the countervailing factors of electrode size, surface characteristics and detection characteristics, certain embodiments of the present invention provide for relatively smaller rough surfaced electrodes having higher sensitivity especially suited for less rapid electroactive compound concentration changes. Still other embodiments provide relatively larger smooth surfaced electrode recording sites especially suited for rapid changes in electroactive compound concentration. For example, pyrolyzed deposited carbon films are generally smoother than typical glassy carbon, i.e., vitreous carbon, used in conventional electrode design. Smoother pyrolyzed carbon-coated ceramic based electrodes of the present invention are believed to provide reduced background noise, facilitate less adsorption of electroactive compounds during in-vivo operation, and provide improved detection and response characteristics than those electrodes currently known in the art.

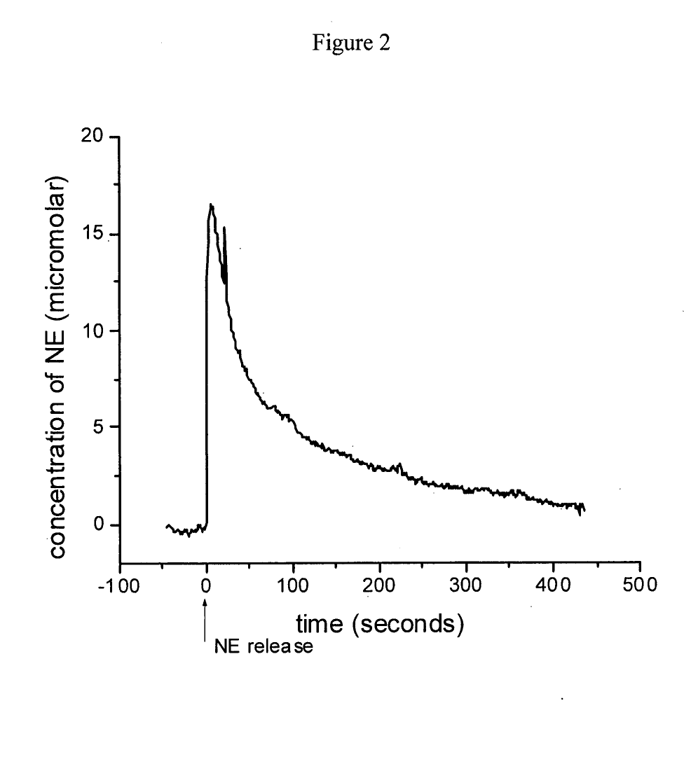

FIG. 2 depicts a graph showing an CCBMSE array embodiment used to detect and measure norepinephrine concentration, as a function of time, in the somatosensory area of a rat brain. Injections of about 58 nL of 2 μM norepinephrine were made into the somatosensory of the rat, wherein the recording site was approximately 150 μm from the injection site. Certain embodiments of the present invention are therefore suited for in-vivo time-dependant neurotransmitter detection and measurement at relatively at least one distant site from neurotransmitter release.

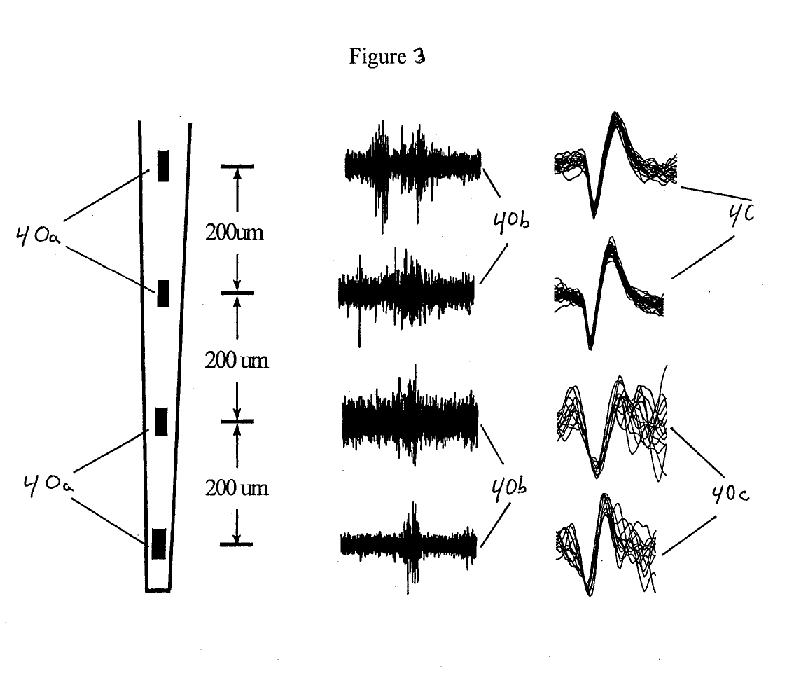

Methods for detecting and/or measuring neurotransmitter concentration in at least two locations of a mammal brain are facilitated by certain embodiments of the present invention. For example, FIG. 3 depicts neural signals simultaneously recorded from four recording sites on the CCBMSE array. Recordings were made from CCBMSE arrays chronically implanted into the somatosensory cortex of rats. Recording sites 40 were spaced about 200 μm along the shaft of the array. Corresponding analog signals 40b were simultaneously recorded from each of the four electrode sites. Still further corresponding individual waveforms 40c were discriminated from the analog signal 40b. Here, certain embodiments of the present invention were shown to be suited for neurotransmitter detection and measurement at multiple locations.

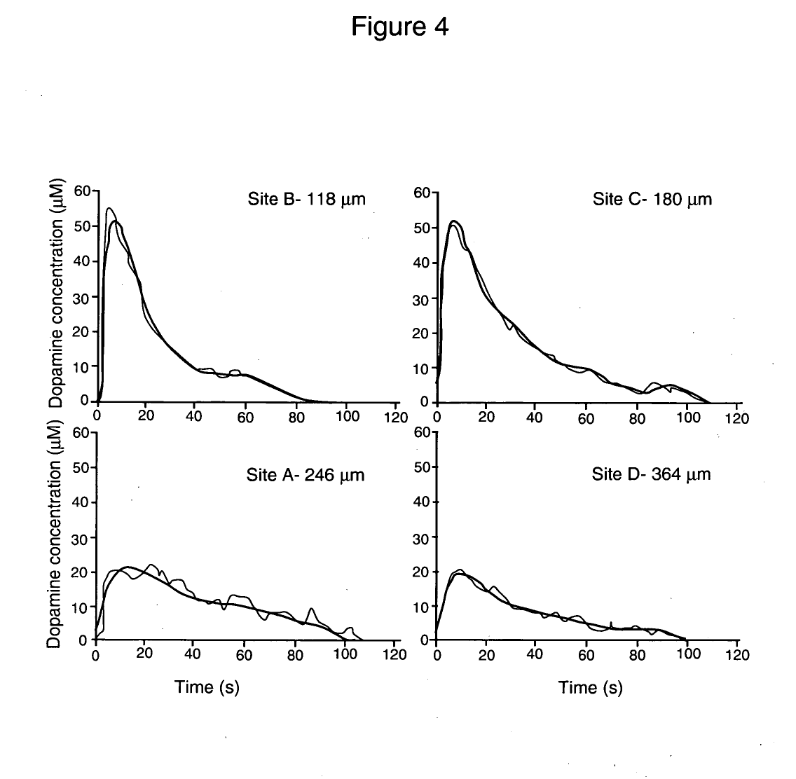

FIG. 4 depicts graphs showing the detection and measurement of dopamine as a function of time and location in the somatosensory area of a rat brain. Injections of about 2 μM dopamine were made to the somatosensory cortex and the resulting diffusion curves were simultaneously measured from four sites distant to the injection site. As noted in the graphs, each respective recording site was 118 μm, 180 μm, 246 μm, and 364 μm from the injection site.

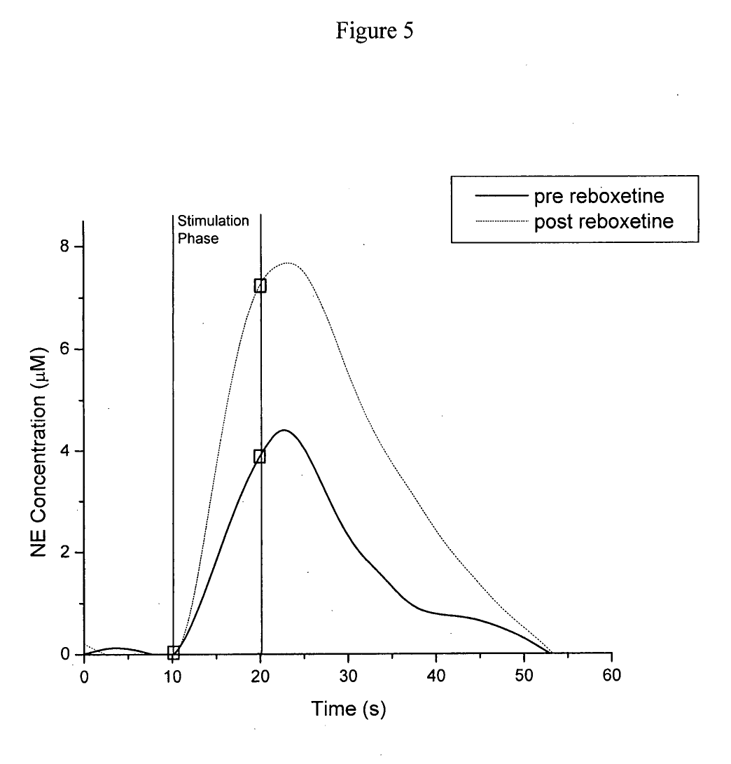

Certain embodiments of the present invention are also suited for in-vivo neurotransmitter detection and measurement in response to drug therapy. FIG. 5 depicts a graph showing in-vivo norepinephrine concentration in response to electrical stimulation of the locus coeruleus (LC) and the effect of a norepinephrine-blocking drug. Here, the LC was electrically stimulated over time (stimulation phase) to effect norepinephrine release. Norepinephrine concentration was measured prior to, during, and after LC stimulation. The solid line represents norepinephrine concentration under normal conditions, e.g., prior to administration of reboxetine—a norepinephrine-reuptake inhibitor. The dotted line represents norepinephrine concentration after administration of the norepinephrine-reuptake inhibitor. Here, drug administration results in higher norepinephrine concentration of because the reuptake-inhibitor slows the removal of norepinephrine from the extracellular space.

Certain embodiments of the present invention provide suitable electrochemical sensors that may simultaneously record the local and global (e.g., micro- and macro-) electrophysiological and electrochemical responses in certain brain regions of interest. Embodiments of the present invention may also be implanted to a mammalián brain, such as a human brain, for ongoing detection and measurement of neurotransmitters of interest. For such applications, at least one chronically implanted, i.e. long term in-vivo implanted, CCBMSE array may act as a sensor in communication with certain other monitoring devices, such as a read-out device and/or recording device. The CCBMSE array output may effect notification, or trigger action, when at least one neurotransmitter of interest is outside a pre-determined range. Such detect and measurement may, for example, signal the onset of loss of drug efficacy. This information could be used to determine when to re-administer certain drugs, adjust drug dosage, or both. Certain embodiments of the present invention are, therefore, useful in the treatment of mental illness such as Parkinson's disease or schizophrenia.

Certain sputter deposited CCBMSE embodiments exhibited a mean root-mean-square (RMS) surface roughness, measured using atomic force microscopy (AFM), more than 10 times greater than for electrodes having untreated platinum surfaces. The RMS roughness for platinum sites was 3.3 nm, while RMS roughness after carbon deposition was about 42.3 nm. Thus, without being limited by theory, one would expect such CCBMSE to exhibit improved detection and measurement of electroactive compounds as compared to conventional platinum electrodes of the same size.

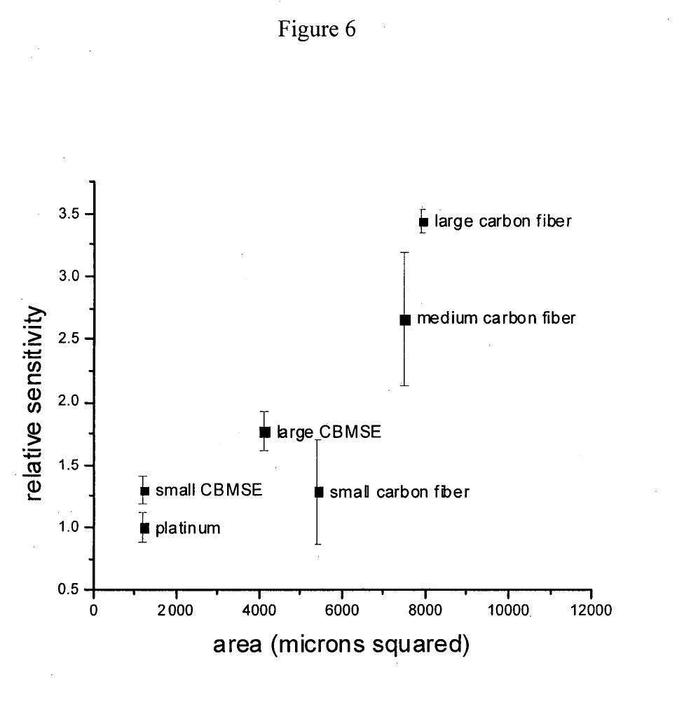

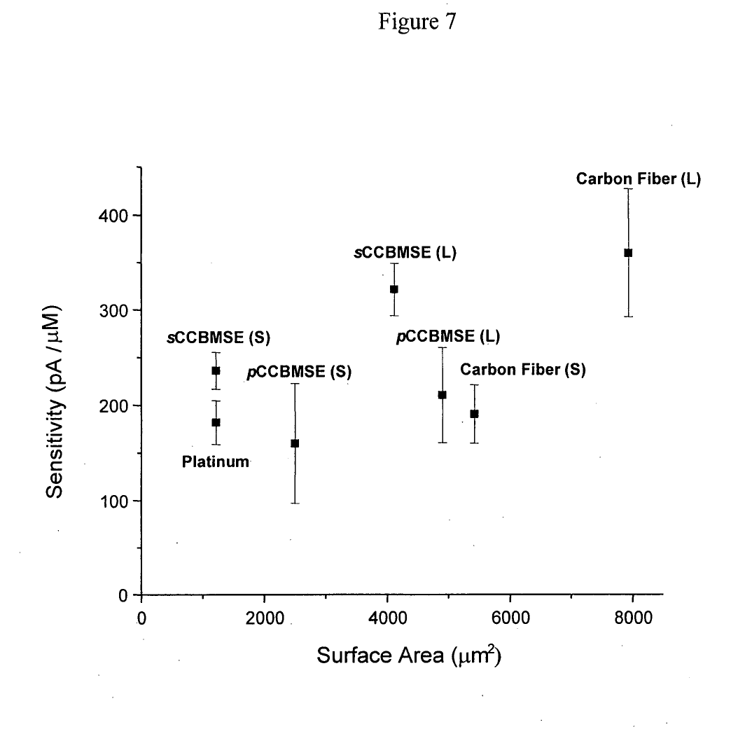

CCBMSE arrays were produced in two different recording site sizes (about 1200 μm² and about 4100 μm²), tested in-vitro and compared to carbon fibers to determine the effect of increased surface roughness and increased recording site size on sensitivity of the electrodes. FIG. 6 shows relative sensitivities for platinum electrodes 51, a small recording site CCBMSE with carbon surface layer (1200 μm²) 52, a large recording site CCBMSE with carbon surface layer (4100 μm²) 53, a small carbon fiber electrode (1200 μm²) 54, a medium carbon fiber electrode (7500 μm²) 55 and a large carbon fiber electrode (7800 μm²) 56. The ion-beam assisted deposition carbon layer showed improved CCBMSE array sensitivity compared to those platinum electrodes known in the art. Still further, sensitivity of the larger CCBMSE array was greater than the smaller carbon fiber electrode. Without being limited by theory, it is contemplated that CCBMSE array embodiments may be “tuned” to further improve sensitivity as compared to larger carbon fibers known in the art, while still minimizing CCBMSE array size. For example, carbon layer deposition may be performed to facilitate increasingly rough surfaces while maintaining recording site size, thus allowing for relatively small recording site having high sensitivity. FIG. 7 depicts a graph showing the sensitivity of relatively small (S) and relatively large (L) pCCBMSE (pyrolized carbon) and sCCBMSE (sputter deposited carbon) arrays as compared to platinum electrodes and conventional carbon fiber microelectrodes of similar size. Generally, both pyrolized and sputter deposition of carbon increased the sensitivity of the CCBMSE arrays compared to platinum alone. sCCBMSE arrays were also generally more sensitive than pCCBMSE arrays at a given size. Without being limited by theory, it is believed that sputtered carbon deposition created a relatively rougher surface having greater effective surface area, and greater sensitivity, than pyrolized carbon surface deposition.

### EXAMPLES

As previously noted, the CCBMSE embodiments of the present invention are especially suited to facilitate improved electrode “tuning” regarding the competing factors of, at least, electrode size, surface characteristics, detection characteristics, and measurement characteristics. Indeed, certain embodiments are suited to balance factors related to sensitivity and response time.

For example, in an in-vivo environment, norepinephrine detection may be improved if such species adsorbs to the surface of the electrode. However, species surface adsorption generally retards microelectrode response time, and in fact may lead to impaired measurement of rapid norepinephrine concentration changes. Still further, electrode sensitivity is generally proportional to electrode size; larger electrode provide greater signal current from the recording site. In-vivo use of large sensors, nonetheless, greatly damage brain tissue.

CCBMSE arrays of the present invention may be tuned according to at least one of, detection characteristics, such as sensitivity and detection limit, and measurement characteristics such as response time and response linearity. Table 1, for example, describes show how CCBMSE electrode size (surface area) and surface roughness may effect certain detection and measurement characteristics.

TABLE 1
Summary of Measured Response Properties

| | Increased | | |
|---|---|---|---|
| | Carbon | Increased | |
| | Surface | Carbon | Determination |
| Properties | Roughness | Surface Area | Method |
| 1. Sensitivity | Increase | Increase | calibration curve |
| | | | slope |
| 2. Limit of detection | Increase | Increase | >3 rms of noise |
| 3. Linearity | Increase | — | calibration curve |
| | | | shape |
| 4. Responsiveness | Decrease | — | flow chamber |
| | | | response |

Items 1-3 were collected during routine electrode calibration and item 4 was evaluated in a flow chamber.

### Electrode Calibration

Calibration curves were generated using the IVEC-10 as shown in FIG. 5. Electrodes were mounted on a frame and connected to the IVEC-10 (In-Vivo Electrochemistry Computer system from Medical Systems Corp., Locust Valley, N.Y.). The CCBMSE array and a reference electrode (Ag/AgCl) were lowered into a beaker with about 40 mL of phosphate buffered saline, pH 7.4. Baseline currents were measured and a gain parameter was set to normalize the background current. Additions of norepinephrine were made in 2 μM increments. The result was a calibration curve whose slope represents the sensitivity of the electrode to the electroactive species; here, the monoamine-norepinephrine. For example, an about 5 micromolar aliquot of monoamine (e.g., 100 μl of 2.0 μM norepinephrine solution) added to the 40 μL beaker increased the concentration by 2 μM. The solution was immediately mixed and approximately 75-100 measurements was taken. At least three successive additions were made to approximate a linear norepinephrine concentration increase. A linear regression analysis of the oxidation and reduction curves was then performed. The oxidation slope of the resultant linear regression analysis represented the calibration factor that, in turn, represented norepinephrine electrode sensitivity. Here, CCBMSE sensitivity greater was generally greater than about 1.25 nA/μM. The limit of detection is defined as the concentration that corresponds to a signal-to-noise level of about 3 times the root-mean square of the spontaneous concentration measurement prior to addition of analyte. Here, the limit of detection was greater than 10 nM. Root-mean-squared noise levels were calculated using 10 points of the baseline current recorded during the calibration procedure. Slope linearity was further indicative of response linearity regarding norepinephrine concentration. Here, linearity was greater than 0.997 for both the oxidation and the reduction current slopes as determined by the Pearson correlation coefficient (R²) of a resultant calibration line.

### Measuring Electrode Response in a Flow Injection Chamber

Responsiveness was measured using the rising slope of the response curves recorded in a flow chamber. Without being limited by theory, embodiments of the present invention are expected to attain a sample rate of at least 1 Hz for electroactive species of interest.

As shown in FIG. 3, an electrode 60 is positioned at the outlet of a flow injection apparatus including a syringe pump 61 (Harvard Apparatus) and rotary valve loop injector 62 made of Teflon (Rheodyne, Inc.) that is mounted on a two-position actuator (Rheodyne model 5041 valve and 5701 actuator). Reference electrode 65 is also included. The syringe pump delivers drug at a flow rate of about 1.0 mL/min. The actuator is used with a 12-V DC solenoid valve kit (Rheodyne) to introduce norepinephrine, dopamine or ascorbic acid through a Teflon tube. The valve is triggered by a computer 63 to turn the loop injector in a rapid and consistent manner. The response of norepinephrine, dopamine or ascorbic acid was recorded by recording device 64 for subsequent analysis.

The temporal difference in the response of the electrode to ascorbic acid (AA) and dopamine (DA) is a measure of adsorption properties of the electrode. Response of the inventive CCBMSE array was compared directly to carbon fiber electrodes. DA and AA have similar chemical structures and redox potentials. However, AA does not adsorb to the electrode surface. Therefore the difference in the curves will be due to adsorption of DA and is a measure of the responsiveness of the electrode.

### Multi-Site Electrode Measurements

Methods for detecting and/or measuring neurotransmitter concentration in at least one location, preferably two locations, of a mammal brain, preferably a human brain, are facilitated by certain embodiments of the present invention. These methods may be further facilitated by use of multi-channel measurement devices, preferably potentiostatic devices, having at least one carbon-coated ceramic based electrode array of the present invention.

For example, it is hypothesized that norepinephrine uptake varies across different layers of the mammalian somatosensory cortex. Accordingly, certain embodiments of the present invention may be used to measure neurotransmitter uptake across different layers of the mammalian brain. In such an experiment, animal subjects are anesthetized and placed in a stereotaxic frame. The skin over the skull is removed and the skull is cleaned. Burr holes are made in the skull for voltammetry electrode, the reference and a working electrode. The recording electrode, having at least one recording site, is placed into the barrel field cortex. At least one neurotransmitter of interest may be administered at a relatively distant location, and concentration changes may be monitored at the recording site(s).

In yet other embodiments, a stimulating electrode is placed into the locus coeruleus. The locus coeruleus efferent path is stimulated using either phasic or tonic patterns of electrical pulses to evoke the release of norepinephrine. For tonic modes, locus coeruleus stimulation frequency would be selected so as to approximate the range of impulse activity that can be maintained physiologically by locus coeruleus axons, i.e., a continuous train of pulses at about 0.5, about 1, about 3, about 6, about 10 or about 20 Hz. The intensity of locus coeruleus stimulation can also be varied across a range of about 0.01 to about 1.0 mA. For phasic mode, short trains (3 pulses/250 msec envelope) of pulses would be delivered to the LC at either about 0.167, about 0.33, about 1.0, about 2.0, about 3.33, or about 6.66 Hz to approximate patterns of phasic bursting of the locus coeruleus across a physiologic range. This frequency and pattern of stimulation may also provide for the delivery of an identical number of pulses to the locus coeruleus during equivalent periods of tonic versus phasic stimulation. Continuous measurements can be made and individual cyclic voltammograms maintained to ensure that the current changes are due to norepinephrine oxidation and not noise or pH shifts. A norepinephrine-specific uptake blocker can be administered to block reuptake and a second set of measurements will be made and compared to the first. Uptake blocker effect can be determined by the peak concentration and time to peak concentration from baseline, time to return to baseline. Thus, the effect local effect of reuptake blockers can be determined during different brain activity levels, e.g., tonic and phasic patterns of locus coeruleus output. By comparing the results of phasic vs. tonic stimulation, before and after reuptake blocking, in multiple layers of the somatosensory cortex simultaneously researchers would have a better understanding of the role of neurotransmitter release and uptake and its impact on brain signal processing.

Certain embodiments of the present invention provide fast-scan multi-channel potentiostatic systems having carbon-coated ceramic based multi-site electrodes and exhibiting improved detection, measurement, or both of electroactive compounds. Certain embodiments further provide fast-scan real-time multi-electrode differential potentiostat systems especially suited to measure levels of electroactive compounds, including neurotransmitters in-vivo and in-vitro, with near simultaneous on-circuit non-Faradayic background removal using multiple microelectrodes. Embodiments of the present invention simultaneously present raw data from multiple electrode channels, and provide at least near simultaneous on-circuit background elimination for the oxidative scan, the reductive scan, or both, as an additional output. Still further embodiments provides a user-chosen driving voltage that controls the potentiostat system. The user-chosen driving voltage may be cyclic, multi-frequency, linear, sine wave, or stepped, or combinations thereof. Such driving voltages may be easily generated and repeated, thus allowing greater flexibility in specifying a driving voltage, scan rate, and repetition rate while maintaining acceptable data acquisition time-lock. The multi-channel potentiostat captures and stores simultaneous voltametric data from multiple microelectrodes with retention of the raw data for all active analysis electrodes. The device rapidly acquires voltammograms at a sampling rate of at least 1 Hz thereby allowing the user to match temporal requirements of the physiological process under investigation.

High-speed multi-electrode differential potentiostat embodiments of the present invention provide potentiostatic control systems using a novel configuration of working electrodes (WEs) as an aggregate control element that develops a single output from a two or four-element Wheatstone bridge. Passive circuit structures such as, but not limited to, a Wheatstone bridge are especially suited to manipulate two or more working electrode signals to effect improved background signal reduction. The amplified floating outputs are combined in a two or four element bridge circuit. Half of the working electrodes are reference and half are sample. The counter electrode (CE) controls the potential between the reference electrode (RE) and aggregate signal from the working electrodes. Common-mode signals from the working electrode cancel while difference signals are transmitted and may be recorded. Such an arrangement, mitigates background (non-faradayic signal) and the effects of common external noise such as powerline and vibration noise. The subtracted channel consists of a signal-rich difference signal and thus poses much less stringent requirements on digitization since the large common background signal may be significantly reduced.

A single-ended-to-differential amplifier (DAx) following a single-ended current-to-voltage converter (CVCx) for each working electrode produces a pair of differential outputs, of the same or nearly identical amplitudes, that are 180 degrees apart in phase. These differential outputs allow for the construction of an active-element Wheatstone-style bridge preferably using only the desired electrode outputs and passive resistors. Such a construction allows for the real-time or near real-time subtraction of said signals with minimal active circuit elements. Additionally, adjustment of the DC voltage offset between each pair of signals, the DC offset of the centerpoint of these two inverses with respect to a fixed potential, and/or the symmetric gain of the pair of signals may be performed, if necessary. The bridge structure generates a subtracted signal as a bridge output. The bridge output can be re-referenced to ground using a second differential amplifier, if desired. Thus, on-circuit analog subtraction at the desired sampling rate may be accomplished without additional active elements. The bridge is preferably constructed of a precision resistor array. The resistor array material may be chosen for low noise and parasitic capacitances that are low enough for the frequencies required by the intended analysis. In this novel application, each pair of signals (one pair from each DAx) and their two types of offsets drive the center of a single arm of the bridge without the application of a conventional excitation voltage to the bridge. The sum of all signal pairs in all arms results in a single subtracted output and the sum of all offsets in all arms results in an analogous single offset. Operation with two DAx's is possible by removing the other two DAx's, grounding the inputs, or replacing the other two DAx's with resistors. Separate gains for each working electrode's differential amplifier allow for very accurate signal matching at the bridge. With the same signal on all electrodes, no output is seen. Thus, the device requires that the sample and reference electrodes either respond differently to the desired electroactive species or are placed in regions of differing concentration of electroactive species. Certain embodiments of the present invention use either two or four working electrodes; however, certain embodiments provide such potentiostat systems having greater than four working electrodes.

Transient electrochemical depletion of electroactive species of interest has been shown as a valid procedure for generating a reference signal. Operating in the bridge-subtraction mode, a preferred reference signal would be one that contains all background and common-mode noise but no signal. Generation of such a reference signal is accomplished by reversibly de-sensitizing the working electrodes to the electroactive species of interest. Such de-sensitizing may be accomplished by oxidizing the working electrode recording sites by oxidation, or like mechanism. The electrodes may then be re-sensitized by an appropriate time/voltage treatment, in order to detect and measure the electroactive species of interest. The electrode may also be desensitized by any number of methods such as, but not limited to, by depleting the local environment of the electroactive species of interest, applying an oxidative coating to the recording sites, or depositing a carbon layer having at least one additive. In this manner, the reference and analysis signals may be obtain without the need to remove or re-insert the working electrodes. Accordingly, certain embodiments of the present invention provide a robust relatively long-term fully implantable CCBMSE array used in conjunction with a fast scan multi-electrode differential potentiostat system.

Oxidation of individual working electrodes, without affecting others, is possible by applying appropriate voltages above or below ground to the input of the current-to-voltage converter for the desired working electrode. This device can detect static levels of electroactive compounds in real-time using fast-scan cyclic voltammetry. Conventional devices are generally insensitive to static concentrations of electroactive compounds due to the need to generate a reference scan that is later subtracted from the sample scan during post-scan analysis. Embodiments of the present invention do not require a reference scan since some or all working electrodes may be rendered insensitive to the electroactive species of interest, and these reversibly de-sensitized working electrodes may thus function as reference electrodes.

The device generates on-circuit background-subtracted voltammetric or amperometric signals, at the sample rate of interest, that require much less bandwidth to digitize and transmit than devices already known in the art. Conventional devices must digitize large background signals along with the desired signal for later removal using a previous or subsequent reference scan. Embodiments of the present invention are especially suited for detecting and measuring rapid changes in electroactive species concentration, as well as static concentrations of species of interest. Still further certain embodiments of the present invention are especially suited for high frequency (fast-scan) detection and measurement at or near the rate of variation for the physiological system(s) of interest. Such analysis may therefore be accomplished in “real-time”. Embodiments further provide for reversible de-sensitizing of individual electrodes.

Embodiments of the present invention are particularly suited for detection and measurement of neurotransmitter levels in a mammalian brain, especially real-time measurements of same. Embodiments are also suited for cyclic voltammetric determination of chemical reaction kinetic and thermodynamic mechanisms, including reaction rate constants. Embodiments of the present invention offer a new degree of freedom in developing improved sensors for electroactive species such as glucose, neurotransmitters, metabolites, pollutants, etc. Indeed, certain embodiments of the present invention may be suitable for electrochemical analytical systems such as, but not limited to, glucose monitoring/control systems, telemetry systems for electroactive species, and water supply analysis systems.

While the invention has been described in detail and with reference to specific examples thereof, it will be apparent to one skilled in the art that various changes and modifications can be made therein without departing from the spirit and scope thereof.

All references cited are incorporated by reference as if fully set forth herein.

What is claimed is:

1. A ceramic based multi-site electrode array comprising:
a ceramic substrate patterned with at least one recording site and at least one bonding pad which are connected via at least one conducting line; and
a ceramic insulating layer encasing said conducting line, wherein said recording site is not encased by said insulating layer, and wherein said recording site comprises at least one layer of carbon.

2. The ceramic based multi-site electrode array of claim 1, wherein said recording site is comprised of at least one layer of platinum, chromium, or titanium, or combinations thereof.

3. The ceramic based multi-site electrode array of claim 1, wherein said insulating layer comprises Al₂O₃.

4. The ceramic based multi-site electrode array of claim 3, wherein said Al₂O₃ is deposited by ion-beam assisted deposition.

5. The ceramic based multi-site electrode array of claim 1, wherein at least a portion of said carbon layer has a surface roughness of at least about 42 rms.

6. A method for detecting or measuring neurotransmitter concentration in at least two locations of a mammal brain comprising implanting said ceramic based multi-site electrode array of claim 1 into a mammal.

7. The ceramic based multi-site electrode array of claim 1 produced by a method comprising:
(a) patterning a ceramic substrate with resist features that define at least one recording site, at least one bonding pad and at least one conducting line connecting said recording site and bonding pad;
(b) depositing at least one metal layer onto the ceramic substrate patterned with the resist features defining said at least one recording site, at least one bonding pads and at least one conducting line;
(c) removing at least a portion of said metal layer such that the remaining metal layer defines at least one recording site, at least one conducting line and at least one bonding pad on said ceramic substrate;
(d) depositing at least one insulating layer to the metal defining said at least one conducting line of the ceramic substrate; and
(e) depositing at least one carbon layer onto said at least one recording site.

8. The method of claim 7, wherein at least a portion of said ceramic substrate is patterned by reverse photolithography.

9. The method of claim 7, wherein said carbon is deposited by sputter deposition, ion-beam assisted deposition, or pyrolysis, or combinations thereof.

10. The method of claim 7, wherein said metal layer removal comprises submersing said ceramic substrate in a photoresist stripper which lifts off at least a portion of an unwanted overlying metal layer.

11. The method of claim 7, wherein said recording site further comprises at least one of platinum, chromium, and titanium.

12. A multi-channel potentiostat device comprising at least one ceramic based multi-site electrode array of claim 1, wherein at least one electrode of the electrode array is a reference electrode; at least one electrode of the electrode array is a working electrode; and said reference and working electrodes are configured as a Wheatstone bridge structure.

13. The multi-channel potentiostat device of claim 12, further comprising a current-to-voltage converter connected to each reference electrode and each working electrode.

14. The multi-channel potentiostat device of claim 13, further comprising an amplifier for producing a separate differential output for all working electrodes.

15. The multi-channel potentiostat device of claim 14, wherein at least two working electrodes contribute to a combined output signal.

16. The multi-channel potentiostat device of claim 12, further comprising an analog to digital converter in communication with said multi-site electrode array.

17. The multi-channel potentiostat device of claim 12, wherein said Wheatstone bridge structure further comprises at least one precision resistor array.

18. The multi-channel potentiostat device of claim 12, wherein at least one working electrode is reversibly de-sensitized to at least one electroactive species of interest.

19. The multi-channel potentiostat device of claim 18, wherein said reversible de-sensitizing comprises oxidation.

20. A method of detecting or measuring electroactive compounds comprising providing at least one carbon coated ceramic based multi-site electrode array of claim 1 having at least one output signal, wherein at least one electrode of said electrode array is a reference electrode and at least one electrode of the electrode array is a working electrode; configuring said at least one output signal into a passive circuit structure; and recording at least one resultant signal from said passive circuit structure.

21. The method of claim 20, wherein said passive circuit structure comprises a Wheatstone bridge structure.
